(19) United States
(12) Patent Application Publication
(10) Pub. No.: US 2026/0224112 A1
(43) Pub. Date: Aug. 6, 2026

(54) METHOD AND SYSTEM FOR ENABLING DETECTION, AND DETECTION OF ATTENTION DEFICIT HYPERACTIVITY DISORDER (ADHD) USING SPECTRAL DATA OF THE BRAIN OF SELECTED BIOCHEMICALS AND MONITORING TREATMENT

(71) Applicants: DatChem, Brisbane (AU); Griffith University, Southport (AU)

(72) Inventors: Carolyn Mountford, Varsity Lakes (AU); David Crompton, Queensland (AU)

(21) Appl. No.: 19/532,530

(22) Filed: Feb. 6, 2026

Related U.S. Application Data
(60) Provisional application No. 63/755,119, filed on Feb. 6, 2025.

(51) Int. Cl. A61B 5/00; A61B 5/16

(52) U.S. Cl. CPC A61B 5/0075; A61B 5/168; A61B 5/4848

(57) ABSTRACT

Adult attention-deficit/hyperactivity disorder (ADHD) is a neuro-developmental disorder which can be detected by obtaining spectral data of at least one biomarker chemical in the brain and comparing the obtained with a reference level of a subject known to not have ADHD to determine whether the subjects obtained levels are different from the reference level. The effect of treatment of a subject having ADHD can also be monitored.

### BACKGROUND OF THE INVENTION

Adult attention-deficit/hyperactivity disorder (ADHD) is a neuro-developmental disorder. In 2020 the worldwide prevalence of persistent adult ADHD was 2.58% and that of symptomatic 6adult ADHD was 6.76%, translating to 139.84 million and 366.33 million affected adults in 2020 globally¹. ADHD is recognized as a disability under the Australian 1992 Disability Discrimination Act and 6-10% of children and 2-6% of adults are diagnosed with ADHD². The economic cost in Australian alone is reported to be $20 billion annually with 800,000 Australians experiencing the disorder³.

ADHD is characterized by procrastination, inattention, difficulty engaging and an inability to focus or prioritize⁴ often impacting an individual's quality of life, hindering ability to complete tasks, manage time, and maintain relationships. Those with ADHD are five times more likely to attempt suicide, and three times more likely to suicide⁵. Males are likely to be diagnosed in childhood whereas females often delayed until adulthood.

Individuals with ADHD symptoms often experience long delays in accessing specialist assessment, diagnosis and treatment with waiting lists of 12 months at a significant economic cost to individuals and country. Psychologists and psychiatrists diagnose ADHD based on the symptoms and a questionnaire-based evaluation with potential biases related to gender, age, symptom type and culture ⁶⁻⁹. ADHD sufferers present with a high level of comorbidity ^{[2]}, symptom overlap with post-traumatic stress disorder (PTSD), major depressive disorder (MDD), anxiety, and substance use disorders ⁶. The present diagnosis methods are highly subjective and often lead to over and under diagnosis errors. There is a need for a more objective and accurate way to diagnose ADHD⁴.

### SUMMARY OF THE INVENTION

The invention provides a method and system for enabling detection and detection of subjects with attention deficit hyperactivity disorder (ADHD) using objective factors, such as selected biomarkers detected from spectral data obtain from a subject's brain.

### BRIEF DESCRIPTION OF THE DRAWINGS

FIG. 1 shows 2D COSY spectra (0-5 ppm) of a subject's brain before and after treatment;

FIG. 2 shows an expanded region of the spectrum (F2:4.1-4.5 ppm, F1:1.1-1.6 ppm) with the fucose-α(1-2)-glycans, fucose-α(1-6)-glycan, and substrate α-L-fucose highlighted;

FIG. 3 shows an aromatic region of the 2D COSY spectra (4.5-8.5 ppm);

FIG. 4 shows Table 1 which summarizes the diagonal and crosspeak values of 2D COSY spectra; and

FIGS. 5A and 5B shows Tables 2A and 2B which summarizes the biochemical levels of selected biochemicals.

### DETAILED DESCRIPTION OF A PREFERRED EMBODIMENT

A preferred embodiment of the present invention will be described, but the present invention is not limited to this embodiment.

In vivo two-dimensional (2D) COrrelated SpectroscopY (2D COSY), accrued in a 3T clinical magnetic resonance (MR) scanner can provide a detailed assessment of an individual's neurochemistry because it unambiguously assigns most molecules in a second magnetic frequency¹⁰. In addition to the well-recorded neurochemical assignments, new multiple fucose-α(1-2)-glycans11 have been assigned in the human brain such as a fucose-α(1-6)-glycan¹² and α-L-Fucose substrates. The fucose-α (1-2)-glycans report on the neuron and its synapse and contribute to plasticity¹³. From animal studies, a growing body of literature implicates these fucose-α (1-2)-glycans in the molecular mechanisms that underlie neuronal development, learning^{14,15}, memory¹⁶ and placticity^{17,18}.

The in vivo 2D COSY protocol provides the capacity to compare an unwell brain with a healthy cohort. This has been successfully implemented for pain^{19,20}, PTSD^{21,22}, blast exposure¹² and determining how the neurochemistry is altered as a function of treatment^{22,23}.

Here, we report a case study of a volunteer patient with a known diagnosis of attention deficit hyperactivity disorder (ADHD). In her early 20s, she was retrospectively diagnosed by a psychiatrist. The diagnosis was based on clinical history and Diagnostic and Statistical Manual of Mental Disorders (DSM 5) criteria Reference: Association, American Psychiatric. 2013. Diagnostic and Statistical Manual of Mental Disorders (DSM-5®). 5th ed. Washington: American Psychiatric Publishing. The assessment included a Diagnostic Interview for ADHD in adults (DIVA-5) . Her medication regime at this time for ADHD was Lisdexamfetamine 50 mg mane.

### Methods

### Volunteer Participants

Clinical assessment was based on DSM 5 criteria. She was assessed using the DIVA-5 24,25 and completed the Depression Anxiety and Stress Scale (DASS 21) ²⁶, Post-traumatic Symptom Checklist (PCL-5) ²⁷, Alcohol Use Disorder Identification Scale (AUDIT 10) ²⁸, adult ADHD self-report scale (ASRS v1.1) ²⁹, the self-report Weiss Functional Impairment Rating Scale (WFIRS-S) ³⁰, Weiss Symptom Record II Form ²⁵, Wender Utah Rating Scale (WURS) ³¹, Generalised Anxiety Disorder Scale (GAD 7) ³², and Patient Health Questionnaire (PHQ 8) ³³. Her partner also completed the ASRS v1.1 and the Weiss Symptom Record II Form for collateral history.

### Comparison to Healthy Controls

The case study data collected were compared with averages collected from a cohort of Healthy Controls (HC) previously reported²¹. The healthy control data were collected from 39 participants aged between 18-65 years with no current DSM-5 Axis I disorder, as assessed by the Structured Clinical Interview for DSM-5 (SCID)³⁴. This control data provided reference levels of biomarkers for healthy subjects.

### MR Imaging

All scans were performed on a 60 cm bore 3T PRISMA scanner (Siemens, Erlangen, Germany, software version VE11C) with a 64-channel head and neck coil (Siemens, Erlangen) at the Herston Imaging Facility (QLD, Australia).

### Structural Imaging

A three plane T2 weighted localizer was performed for volume of interest placement. For anatomical morphometry and voxel placement a three-dimensional T1 weighted MPRAGE³⁵ was acquired (TR/TE=2530/1.7ms, 12⁰ flip angle, FOV=256×256 mm, voxel size 1 mm³, NEX 4, IPAT=3, acquisition time=4 mins), for accurate MRS voxel placement.

### 2D L-Cosy Acquisition and Analysis

A 2D L-COSY spectrum was acquired from a 3 cm³ voxel located in the PCG. RF carrier frequency was set at 2.4 ppm, TR 1.5 second; water suppression using WET; spectral width of 2000 Hz; with 96 T 1 increments, increment size of 0.8 ms in (giving an indirect spectral width of 1250 Hz); 8 averages per increment and 1024 data points¹¹. Acquisition time was 19 minutes. Shimming adjustments were undertaken on each scan by invalidating the automatic B0 field mapping technique supplied by the vendor before rerunning the automatic shim and manually adjusting the shim gradients in the X, Y and Z directions to achieve a full width half maximum (FWHM) of the water peak between 12-15 Hz.

All raw data were pre-processed as reported previously ^{11,23,36}. Each prominent diagonal and cross peak was selected and integrated to determine the peak chemical shift, intensity, and volume. These values were internally referenced using the total creatine methyl diagonal peak at 3.02 ppm.

### Results

Clinical Evaluation: The volunteer patient presented with ADHD and this was confirmed by an independent psychiatrist (DC). Her ADHD medication regime at this time was Lisdexamfetamine 50 mg mane. Additionally, she is prescribed Candesartan 4 mg mane for hypertension, Clonidine 100 mg nocte for migraines and Escitalopram 10 mg mane for anxiety and depressive symptoms. The patient also reported symptoms indicative of endometriosis. The patient's school reports were reviewed, indicating that she performed well academically and achieved an OP4 ranking in her final year. She graduated from University with a BA, majoring in English literature and film. Despite her academic performance, school reports commented on inappropriate behavior in the classroom and being easily distracted. Some teachers noted she tended to waste time chatting and had difficulties with time management.

The clinical history indicates that she was diagnosed with depression at the age of 15, despite treatment for depression and associated anxiety symptoms, she continued to experience several symptoms, including difficulties with social interactions and completing and attending to tasks. In addition, she reported a tendency to not follow instructions, to lose focus and to be distracted by external stimuli. She described a tendency to restlessness and complete other people's sentences. The symptoms became more apparent after school and impacted her work, domestic life and friendships. The continued symptoms resulted in her seeking a psychiatric assessment in her 20s, leading to the diagnosis of ADHD. The clinical history using DSM 5 criteria supports a past diagnosis of major depression and comorbid anxiety. There is no evidence of alcohol or substance use disorders. Nor is there a history of trauma.

The DIVA-5 evaluation supports the current diagnosis of ADHD with inattention as the predominant feature. The evaluation indicates the symptoms were evident in childhood. The psychological assessments were completed when she was taking Lisdexamfetamine and Escitalopram. The ASRS v1.1 score was below the cut-off score for ADHD. However, the Weiss Symptom Record II assessment (score 2.2) was consistent with impairment related to difficulties with attention. The WFIRS-S identified impairment of life skills and while the WURS score did not suggest impairment in childhood, the assessment indicated problems with distraction, concentration, daydreaming and failing to finish tasks.

The GAD 7 and PHQ 8 suggested the presence of mild anxiety and depressive symptoms. However, the DASS 21 did not indicate the presence of clinically significant psychological distress, depression or anxiety. The PCL score was not consistent with a diagnosis of PTSD. The OHIO Traumatic Brain Inventory assessment REF identified a fall around the age of two; however, there was no loss of consciousness and no other evidence of traumatic brain injury since that time. Her partner's ASRS v1.1 and Weiss Symptom Record II Form assessments were similar to those recorded by the patient.

Based on DSM 5 criteria, the patient fulfils the criteria for ADHD attention deficit type and comorbid anxiety and depressive symptoms. Despite treatment, there is an indication of ongoing symptomatology and impairment of her self-reported life-skills functioning.

The patient recommenced medication 24 hours after the initial clinical MR scan and the scan within repeated six hours of taking the Lisdexamfetamine 50 mg.

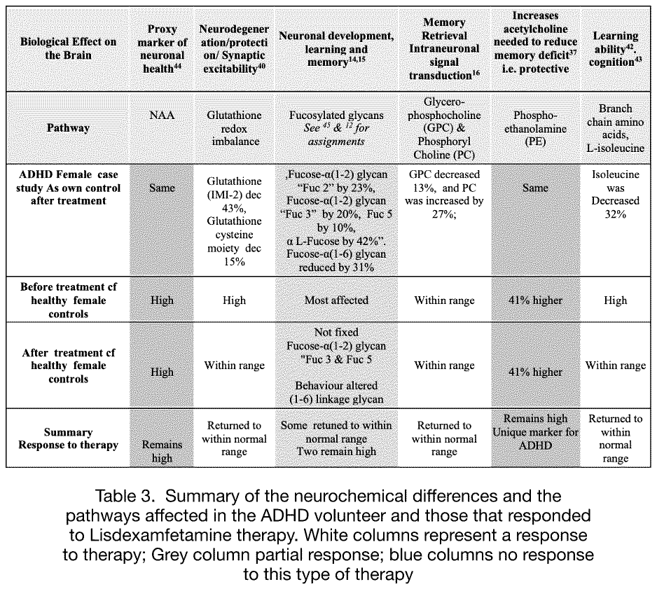

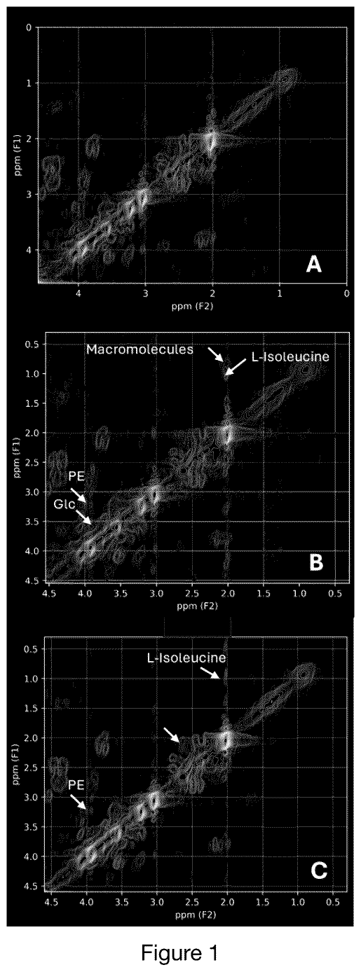

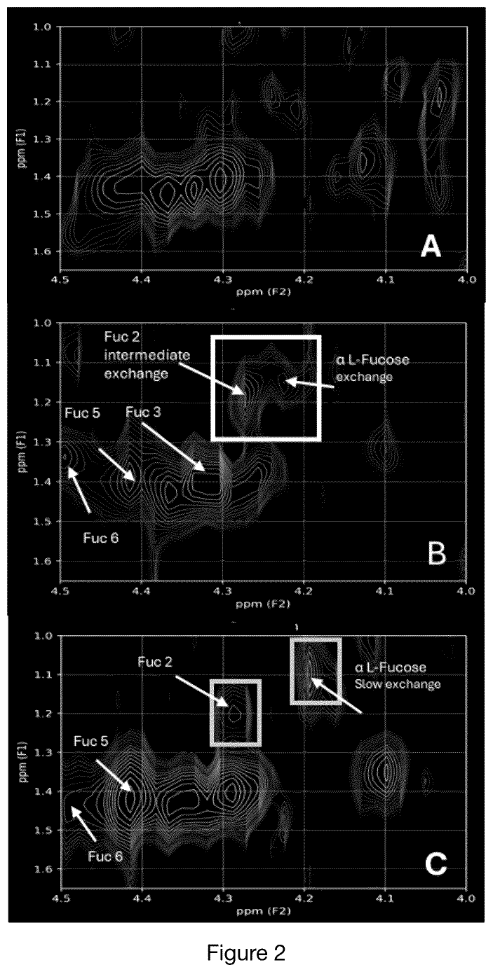

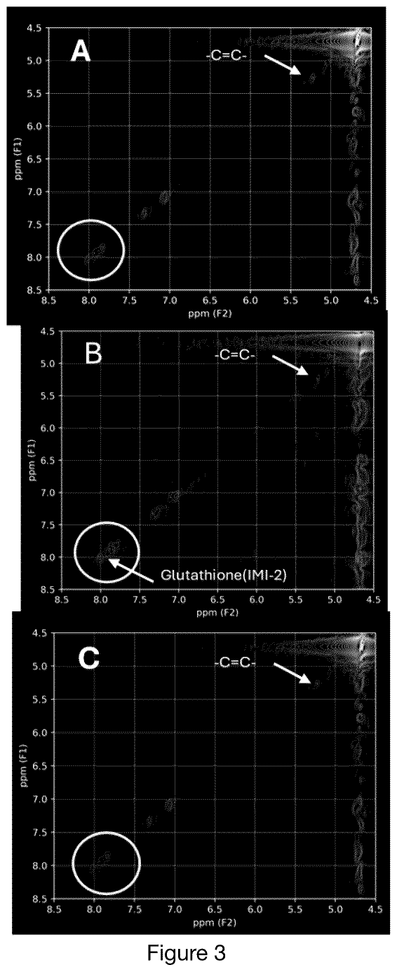

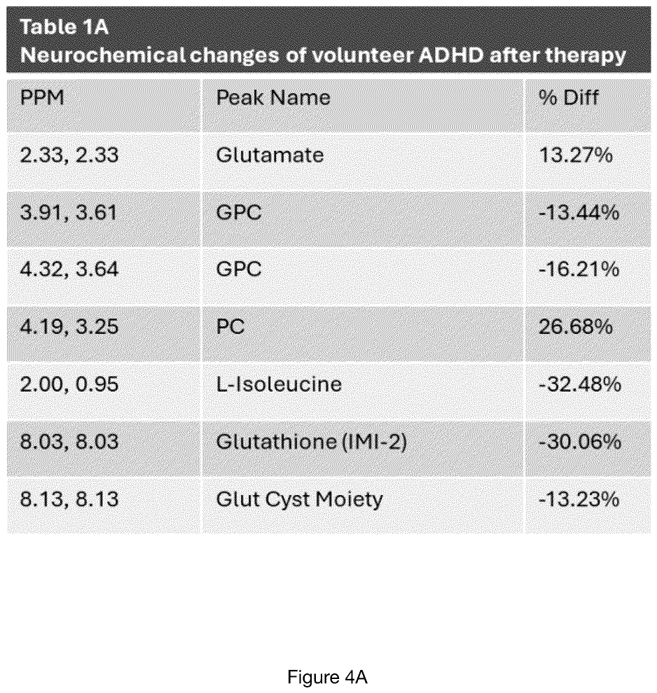

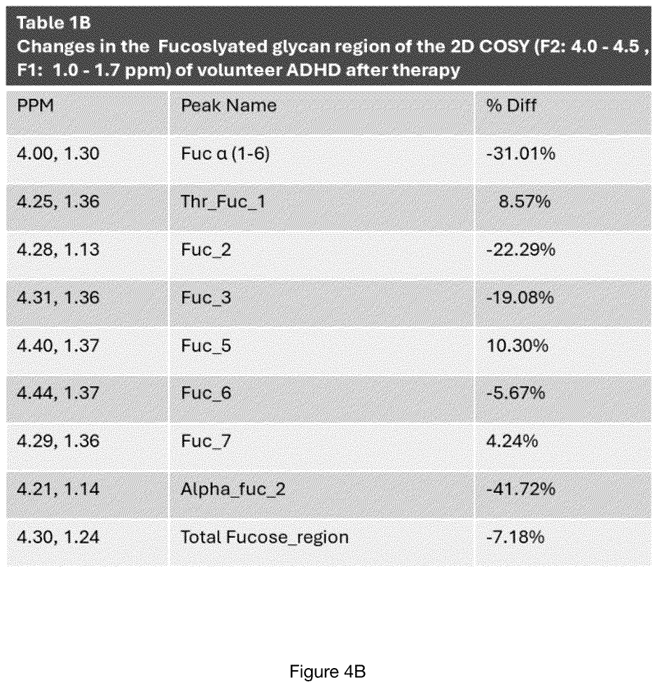

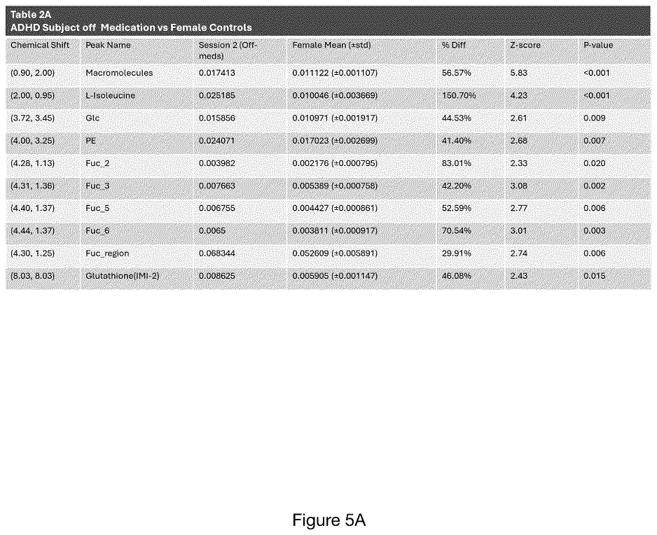

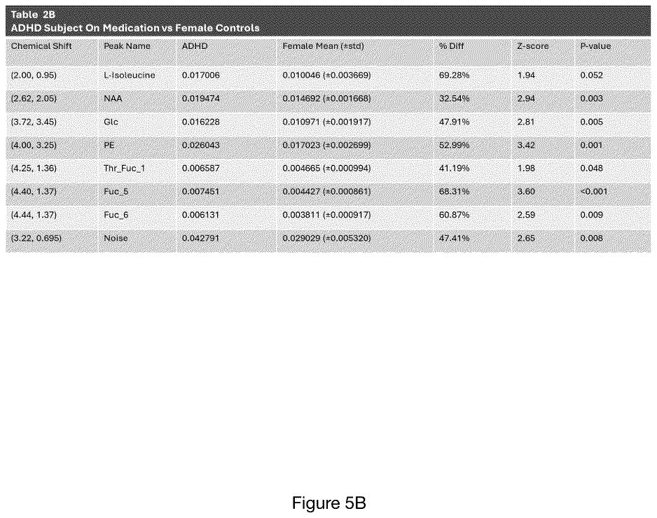

Neurochemistry Evaluated by 2d Cosy: the 2D COSY Spectra (0-5 ppm) before and after treatment are shown in FIG. 1. The expanded region of the spectrum (F2:4.1-4.5 ppm, F1:1.1-1.6 ppm) are shown in FIG. 2 with the fucose-α(1-2)-glycans, fucose-α(1-6)-glycan and substrate α-L-fucose highlighted The aromatic region of the 2D COSY spectra (4.5-8.5 ppm) are shown in FIG. 3.

FIG. 1 shows in Vivo L-COSY of the human brain (Post Cingulate Gyrus) acquired at 3T (Prisma) using a 64-channel head and neck coil; voxel size 30×30×30 mm³, increment size 0.8 ms, increments 96, 8 averages per increment, TR 1.5 sec, total experimental time 19.12 min, acquired vector: 1024 points, acquisition time: 512 ms, spectral width in F2: 2000 Hz, spectral with in F1:1250 Hz.
A. Healthy female control same age.
B. Volunteer ADHD patient off medication for 24 hours.
C. Patient on Lisdexamfetamine therapy.

Assignments are as in Mountford et al⁴⁵.

FIG. 2 shows expanded region of the L-COSY spectrum in FIG. 1 (F2: 4.1-4.5 ppm, F1:1.1-1.6 ppm), with the assignments of the fucose-α(1-2)-glycans, Fuc 1 to 7 denoted^{11,45}. The Fucose-α(1-2) glycan “Fuc 2” changes from intermediate exchange with the substrate to slow exchange after treatment.
A. Healthy control
B. ADHD Patient no therapy
C. ADHD Patient on Lisdexamfetamine therapy

FIG. 3 shows expanded region of the L-COSY spectrum (F2: 4.5-9.0 ppm, F1:4.5-9.0 ppm), with the assignment of and the lipid C=C at 5.23 ppmm and Glutathione(IMI-2) at 8.05 ppm. The Glutathione(IMI-2) was reduced by 13.23% when the patient treated with Lisdexamfetamine therapy.
A. Healthy control
B. Patient no therapy
C. Patient on therapy.

Table 1A shows the difference between neurochemicals, involved in the pathways associated with measured using in vivo 2D L-COSY of an ADHD patient pre-therapy, post-therapy. Patient peak volumes are referenced to creatine.

Table 1B shows changes in the Fucoslyated glycan region of the 2D COSY (F2: 4.0-4.5, F1:1.0-1.7 ppm) of volunteer ADHD after therapy

### Comparison of Adhd Volunteer Before and After Therapy As her Own Control

The Diagonal and crosspeak values of the 2D COSY spectra are summarised in Table 1 before

and after treatment. From this table it can be seen that the ADHD patients'glutathione (IMI-2) (F2:2.28 ppm, F1:2.00 pm) level have increased by 43% the glutamine cysteine level (F2: 2.28 ppm, F1:2.00 pm) by 15%. GPC and PC (F2:4.19 ppm, F1:3.25ppm) levels were reduced by 17% and 21%, respectively L-isoleucine (F2:2.00 ppm, F1:0.95 ppm) showed an increase of 48%. There was a small increase in phenylalanine of 3% and GABA reduced by 2%.

In the spectral region, the COSY cross peak intensities of fucoslyated glycans shown substantive differences after treatment. Evaluation of the fucose-α(1-6) glycan (F2: 4.00 ppm, F1:1.30ppm) showed an increase of 45%; Fucose-α(1-2) glycan “Fuc 2” by 29% (F2: 4.28 ppm, F1:1.13 ppm); Fucose-α(1-2) glycan “Fuc 3” by 34% (F2: 4.31 ppm, F1:1.16 ppm). The Fucose-α(1-2) glycan “Fuc 5” (F2 : 4.40 ppm, F1:1.37 ppm) decreased by 9% and Fucose-α(1-2) glycan “Fuc 7” (F2: 4.29 ppm, F1:1.36 ppm) decreased by 4 (Table 2.).

In the untreated brain, both the Fucose-α(1-2) glycan ‘Fuc 2’ (chemical shifts F2: 4.28 ppm, F1: 1.13 ppm) and its substrate αL-Fucose appear to exhibit slow exchange on the MR timescale. This could indicate either limited interaction or slow conformational changes between the glycan and its substrate under normal conditions. After treatment, the exchange rate increases to an intermediate regime (as shown in FIG. 2), suggesting that the treatment accelerates the dynamics between Fucose-α(1-2) glycan ‘Fuc 2’ and αL-Fucose.

The untreated ADHD brain recorded no homocarnosine-GABA nor glutathione cysteine. After treatment the ADHD patient's glutathione (IMI-2) increased 43%, glutamine cysteine moiety increased 15%; glycerophosphocholine (GPC) increased 17%; phosphoryl choline (PC) reduced by 20%; L-isoleucine increased 48%; Fucose-α(1-6) glycan increased 46%, Fucose-α(1-2) glycans “Fuc 2” and “Fuc 3” increased by 29% and 34%, respectively; and α L-Fucose increased by 72%”.

Compared to the controls the untreated ADHD brain recorded considerably higher phosphoethanolamine (PE) (41%) L-isoleucine (151%), glucose (44%), PE (41%), glutathione(IMI-2) (46%) and Fucose-α(1-2) glycans, “Fuc 2” 83%; “Fuc 3” 42%; “Fuc 5”53% and “Fuc 6” 71%. Following treatment, compared to controls, L-isoleucine, glutathione (IMI-2) and fucoslyated (1-2) glycan “Fuc 3” returned to the healthy range. However, PE, fucoslyated (1-2) glycans “Fuc 5” and “Fuc 6” and glucose remained high.

Lisdexamfetamine corrected pathways linked to neurodegeneration and synaptic excitability, memory retrieval and some of the Fucose-α(1-2) glycans involved in plasticity and cognition. The treatment failed to correct pathways linked to neuronal health; the fucose-α(1-2) glycans “Fuc 5” and “Fuc 6”; and PE related to memory. An objective, biomarker-based evaluation for ADHD is now be feasible, providing an objective diagnosis and ability to monitor the effectiveness of targeted therapy.

Comparison of ADHD Volunteer Before Therapy With The Female Healthy Control Database

The diagonal and crosspeak values that were measured are summarised in Table 2 where the average value for the healthy cohort is compared with the patients'data at each timepoint. Included in FIGS. 1-3 are an age and gender matched healthy control for comparison.

Prior to treatment the concentration of nine neurochemicals associated with brain activity were considerably higher compared to the healthy control database (Table 2). Following treatment the macromolecules, L-Isoleucine, glutathione (IMI-2) and fucoslyated (1-2) glycan “Fuc 3” were in the healthy range. Phosphoethanolamine remained high as did fucoslyated (1-2) glycan “Fuc 5” and “Fuc 6”. Glucose (Glc) remained high. Following treatment, the NAA levels increased compared to the healthy cohort.

### Discussion

These data demonstrate that the 2D COSY method can monitor the effect of therapy on the ADHD brain and how the ADHD brain of this patient differed to a cohort of control females with and without therapy. There are multiple interesting and potentially important findings.
1. High levels of PE was recorded in the ADHD brain, and it was not affected by treatment. The literature suggests high levels of PE increases acetylcholine needed to reduce memory deficit 37 i.e. is protective. Also, others suggest that α-Glyceryl-phosphoryl-ethanolamine protects human hippocampal neurons from ageing-induced cellular alterations³⁷. Recent reports suggest that individuals with abnormal PE levels suffer from multisystem disorders mainly affecting neuronal function³⁸. Phosphatidylethanolamine (PE) is a phospholipid that's found in the brain and other nervous tissue. It's involved in many biological processes, including membrane fusion, protein folding, and lipid metabolism. PE is a precursor to N-acylethanolamine, a neurotransmitter in the brain. PE helps provide acetylcholine, which can help reduce memory deficits³⁷.
2. Following treatment compared to the untreated brain the L-isoleucine level decreased by 49%. Compared to the healthy cohort the untreated ADHD brain was 150% higher and after treatment was only 62% higher. L-isoleucine is an amino acid that's transported into the brain and used to synthesize neurotransmitters. It's one of three essential amino acids that make up branched-chain amino acids (BCAAs), along with leucine and valine³⁹.
3. A glutathione redox imbalance is linked to neurodegeneration, protection and synaptic excitability⁴⁰. Following treatment the Glutathione (IMI-2) decreased by 43%, and the glutathione cysteine moiety decreased by 15% compared to the untreated self-control brain. When compared to the healthy female cohort the pretreatment ADHD brain recorded Glutathione (IMI-2) 46% higher and returned to the normal region post treatment.
4. The untreated ADHD brain demonstrated what appeared to be an intermediate exchange between the fucose-α(1-2)-glycan, denoted “Fuc 2”, (F:2 4.28, F1:1.14 ppm) crosspeak and the substrate α-L-fucose at (F:2 4.22, F1:1.15 ppm). The ADHD treated brain recorded a slow exchange where the Fuc 2”, (F:2 4.28, F1:1.12 ppm) crosspeak and the substrate α-L-fucose at (F:2 4.22, F1:1.10 ppm) (FIG. 2). We are not aware of a kinetic exchange rate being recorded in the human brain before for the terminal fucose-α(1-2)-glycans with a substrate.

The Hsieh-Wilson model suggests potentially important roles for fucose-α(1-2)Gal sugars in the regulation of the neurons and morphological changes that may underlie synaptic plasticity in the brain⁴¹. These results support the suggestion that the appropriate treatment this type of plasticity can be treatable. The branched chain amino acids are thought to be involved in learning ability and cognition^{42,43}.

Lisdexamfetamine, a prodrug converted into dextroamphetamine in the body, increases the levels of dopamine and norepinephrine in the brain targeting the dopamine transporter (DAT) and norepinephrine transporter (NET), reversing their action to enhance neurotransmission. The elevated levels of dopamine and norepinephrine may influence gene expression and enzymatic activities responsible for fucose metabolism, such as those involving fucosyltransferases, fucosidases and GDP-Fucose synthase. These changes could alter the glycosylation patterns of synaptic proteins, affecting neuronal function and signaling pathways. Therefore, the therapeutic effects and our observed changes in the brain chemistry following treatment might be partly due to its impact on fucose metabolism, offering new insights into its efficacy and potential avenues for more targeted ADHD therapies.

A patient with ADHD demonstrates five neurochemical pathways (NAA, glutathione redox imbalance, GPC and PC; phosphoethanolamine; branched chain amino acid L-isoleucine; and three fucose-α(1-2)-glycans and one fucose-α(1-6)-glycan were all considerably higher compared to a healthy age and gender matched control cohort. The effect of Lisdexamfetamine (24 hours after taken) corrected the glutathione redox imbalance; the fucose-α(1-6)-glycan level; the GPC and PC level; and the branched chain amino L-isoleucine levels. Lisdexamfetamine did not correct the high levels of NAA nor phosphoethanolamine. These remain high. The present invention provides a method and system to diagnose and monitor treatment for ADHD and assist in design directed therapies.

### REFERENCES CITED, WHICH ARE INCORPORATED BY REFERENCE HEREIN

1. Song P, Zha M, Yang Q, Zhang Y, Li X, Rudan I. The prevalence of adult attention-deficit hyperactivity disorder: A global systematic review and meta-analysis. J Glob Health 2021; 11: 04009.
2. May T, Birch E, Chaves K, et al. The Australian evidence-based clinical practice guideline for attention deficit hyperactivity disorder. Aust N Z J Psychiatry 2023; 57(8): 1101-16.
3. Chen H, Yang Y, Odisho D, Wu S, Yi C, Oliver B G. Can biomarkers be used to diagnose attention deficit hyperactivity disorder? Front Psychiatry 2023; 14:1026616.
4. Drechsler R, Brem S, Brandeis D, Grönblatt E, Berger G, Walitza S. ADHD: Current Concepts and Treatments in Children and Adolescents. Neuropediatrics 2020; 51(5): 315-35.
5. Fuller-Thomson E, RiviÈre R N, Carrique L, Agbeyaka S. The Dark Side of ADHD: Factors Associated With Suicide Attempts Among Those With ADHD in a National Representative Canadian Sample. Archives of Suicide Research 2022; 26(3): 1122-40.
6. Williamson D, Johnston C. Gender differences in adults with attention-deficit/hyperactivity disorder: A narrative review. Clinical psychology review 2015; 40: 15-27.
7. Young J L, Goodman D W. Adult attention-deficit/hyperactivity disorder diagnosis, management, and treatment in the DSM-5 Era. Primary care companion for CNS disorders 2016; 18(6).
8. Belirgan S, Ersoy M A, Topçu Ersoy H. Prevalence of adult attention deficit hyperactivity disorder and comorbid axis-I disorders among first time applied cases of a general psychiatry outpatient clinic and a private psychotherapy centre. Klinik psikofarmakoloji bülteni 2018; 28(1): 25-35.
9. Daniel H M C, Duarte I, Caye A, Rohde L A. Exploring Diagnostic Performance of a Screening Instrument for ADHD and DSM-5 Number of Symptoms Criterion in Primary School Students From Mozambique. Journal of attention disorders 2024; 28(5): 583-8.
10. Thomas M A, Hattori N, Umeda M, Sawada T, Naruse S. Evaluation of two-dimensional L-COSY and JPRESS using a 3T MRI scanner: from phantoms to human brain in vivo. NMR in biomedicine 2003; 16:245-51.
11. Tosh N, Quadrelli S, Galloway G, Mountford C. Two New Fucose-α (1-2)-Glycans Assigned In The Healthy Human Brain Taking The Number To Seven. Scientific Reports 2019; 9(1): 18806.
12. Tosh N, Watson J, Lukas D, et al. Two-dimensional correlated spectroscopy records reduced neurotransmission in blast-exposed artillery soldiers after live fire training. NMR in biomedicine 2023; 36(9): e4934.
13. Murrey H E, Ficarro S B, Krishnamurthy C, Domino S E, Peters E C, Hsieh-Wilson LC. Identification of the Plasticity-Relevant Fucose-α (1 2)-Galactose Proteome from the Mouse Olfactory Bulb. Biochemistry 2009; 48(30): 7261-70.
14. Murrey H E, Ficarro S B, Krishnamurthy C, Domino S E, Peters E C, Hsieh-WilsonLC. Identification of the Plasticity-Relevant Fucose-α(1−2)-Galactose Proteome from the Mouse Olfactory Bulb. Biochemistry 2009; 48(30): 7261-70.
15. Murrey H E, Hsieh-Wilson L C. The chemical neurobiology of carbohydrates. Chemical Reviews 2008; 108(5): 1708-31.
16. Polis B, Samson A. Role of the metabolism of branched-chain amino acids in the development of Alzheimer's disease and other metabolic disorders. Neural Regeneration Research 2020; 15:1460-70.
17. Gu W, Fukuda T, Isaji T, et al. Loss of & #x3b 1; 1,6-Fucosyltransferase Decreases Hippocampal Long Term Potentiation: IMPLICATIONS FOR CORE FUCOSYLATION IN THE REGULATION OF AMPA RECEPTOR HETEROMERIZATION AND CELLULAR SIGNALING *. Journal of Biological Chemistry 2015; 290(28): 17566-75.
18. Murrey H E, Ficarro S B, Krishnamurthy C, Domino S E, Peters E C, Hsieh-Wilson LC. Identification of the plasticity-relevant fucose-alpha(1-2)-galactose proteome from the mouse olfactory bulb. Biochemistry 2009; 48(30): 7261-70.
19. Siddall P, Mountford, C. et al, inventor System and method for detecting pain and its components using magnetic resonance patent 20100152569. 2014.
20. Siddall P J, Stanwell P, Woodhouse A, et al. Magnetic resonance spectroscopy detects biochemical changes in the brain associated with chronic low back pain: a preliminary report. Anesthesia and analgesia 2006; 102(4): 1164-8.
21. Quadrelli S, Tosh N, Urquhart A, et al. Post-traumatic stress disorder affects fucose-α (1-2)-glycans in the human brain: preliminary findings of neuro deregulation using in vivo two-dimensional neuro MR spectroscopy. Translational Psychiatry 2019; 9(1): 27.
22. Tosh N C, David, et al. Serendipitous neurotransmitter findings in a frontline defender participating in a magnetic resonance spectroscopy study of first responders and military personnel with PTSD. Journal of Clinical Case Reports, Medical Images and HealthSciences 2023; 6(1).
23. Watson J, Lukas D, Vickers E R, Galloway G, Mountford C E. Case Report: Capacity to Objectively Monitor the Response of a Chronic Pain Patient to Treatment. Frontiers in Neuroimaging 2022; 1.
24. Zamani L, Shahrivar Z, Alaghband-Rad J, et al. Reliability, criterion and Concurrent validity of the Farsi translation of DIVA-5: a semi-structured diagnostic interview for adults with ADHD. Journal of attention disorders 2021; 25(12): 1666-75.
25. Weiss M D, Stein M A. Measurement informed care in attention-deficit/hyperactivity disorder (ADHD). Child and adolescent psychiatric clinics of North America 2022; 31(3): 363-72.
26. Coker A O, Coker O, Sanni D. Psychometric properties of the 21-item depression anxiety stress scale (DASS-21). African Research Review 2018; 12(2): 135-42.
27. Blevins C A, Weathers F W, Davis M T, Witte T K, Domino J L. The posttraumatic stress disorder checklist for DSM-5 (PCL-5): Development and initial psychometric evaluation. Journal of traumatic stress 2015; 28(6): 489-98.
28. Saunders J B, Aasland O G, Babor T F, De la Fuente J R, Grant M. Development of the alcohol use disorders identification test (AUDIT): WHO collaborative project on early detection of persons with harmful alcohol consumption-II. Addiction 1993; 88(6): 791-804.
29. Silverstein M J, Faraone S V, Alperin S, Biederman J, Spencer T J, Adler LA. How Informative Are Self-Reported Adult Attention-Deficit/Hyperactivity Disorder Symptoms? An Examination of the Agreement between the Adult Attention-Deficit/Hyperactivity Disorder Self-Report Scale V1.1 and Adult Attention-Deficit/Hyperactivity Disorder Investigator Symptom Rating Scale. Journal of child and adolescent psychopharmacology 2018; 28(5): 339-49.
30. Canu W H, Hartung C M, Stevens A E, Lefler E K. Psychometric Properties of the Weiss Functional Impairment Rating Scale: Evidence for Utility in Research, Assessment, and Treatment of ADHD in Emerging Adults. Journal of attention disorders 2020; 24(12): 1648-60.
31. Lundervold A J, Vartiainen H, Jensen D, Haavik J. Test-Retest Reliability of the 25-item version of Wender Utah Rating Scale. Impact of Current ADHD Severity on Retrospectively Assessed Childhood Symptoms. Journal of attention disorders 2021; 25(7): 1001-9.
32. Jordan P, Shedden-Mora M C, Löwe B. Psychometric analysis of the Generalized Anxiety Disorder scale (GAD-7) in primary care using modern item response theory. PloS one 2017; 12(8): e 0182162-e.
33. Dancyger C, Kelleher K, Barrington M. A patient centred approach to measuring outcomes in psycho-oncology services: The PHQ-8 as a viable alternative to the PHQ-9. Psycho-oncology (Chichester, England) 2022; 31(3): 548-50.
34. First M B, Spitzer, Robert L, Gibbon Miriam, and Williams, Janet B. W. Structured Clinical Interview for DSM-IV-TR Axis I Disorders, Research Version, Patient Edition (SCID-I/P). New York: New York State Psychiatric Institute; 2002.
35. Mugler J P, 3rd, Brookeman J R. Three-dimensional magnetization-prepared rapid gradient-echo imaging (3D MP RAGE). Magn Reson Med 1990; 15(1): 152-7.
36. Tosh N, Crompton, D., Tremewan, R., Beard, J., Galloway, G., Young, R., & Mountford, C. Serendipitous neurotransmitter findings in a frontline defender participating in a magnetic resonance spectroscopy study of first responders and military personnel with PTSD. Journal of Clinical Case Reports, Medical Images and Health Sciences 2023; 6.
37. Zappelli E, Daniele S, Ceccarelli L, et al. α-Glyceryl-phosphoryl-ethanolamine protects human hippocampal neurons from ageing-induced cellular alterations. Eur J Neurosci 2022; 56(5): 4514-28.
38. St Germain M, Iraji R, Bakovic M. Phosphatidylethanolamine homeostasis under conditions of impaired CDP-ethanolamine pathway or phosphatidylserine decarboxylation. Front Nutr 2022; 9:1094273.
39. Salcedo C, Andersen J V, Vinten KT, et al. Functional Metabolic Mapping Reveals Highly Active Branched-Chain Amino Acid Metabolism in Human Astrocytes, Which Is Impaired in iPSC-Derived Astrocytes in Alzheimer's Disease. Frontiers in Aging Neuroscience 2021; 13.
40. Gu F, Chauhan V, Chauhan A. Glutathione redox imbalance in brain disorders. Current Opinion in Clinical Nutrition & Metabolic Care 2015; 18(1): 89-95.
41. Ashe K, Kelso W, Farrand S, et al. Psychiatric and Cognitive Aspects of Phenylketonuria: The Limitations of Diet and Promise of New Treatments. Front Psychiatry 2019; 10:561-.
42. Shida Y, Endo H, Owada S, et al. Branched-chain amino acids govern the high learning ability phenotype in Tokai high avoider (THA) rats. Scientific Reports 2021; 11(1): 23104.
43. Xing G, Ren M, Verma A. Divergent Induction of Branched-Chain Aminotransferases and Phosphorylation of Branched Chain Keto-Acid Dehydrogenase Is a Potential Mechanism Coupling Branched-Chain Keto-Acid-Mediated-Astrocyte Activation to Branched-Chain Amino Acid Depletion-Mediated Cognitive Deficit after Traumatic Brain Injury. J Neurotrauma 2018; 35(20): 2482-94.
44. Rebelos E, Daniele G, Campi B, et al. Circulating N-Acetylaspartate does not track brain NAA concentrations, cognitive function or features of small vessel disease in humans. Scientific Reports 2022; 12(1): 11530.
45. Mountford C, Quadrelli S, Lin A, Ramadan S. Six fucose-alpha(1-2) sugars and alpha-fucose assigned in the human brain using in vivo two-dimensional MRS. NMR Biomed 2015; 28(3): 291-6.

1. A method for enabling detection of a subject to have attention-deficit/hyperactivity disorder (ADHD), comprising:
obtaining spectral data of the brain of a subject for at least one of the following biochemicals:
glutathione,
glutamine cysteine moiety,
glycerophosphocholine,
phosphoryh choline (PC),
L-isoleucine,
fucose α (1-6) glycans,
fucose α (1-2) glycan,
A phophoethanolamine,
glucose, and
Polyethylene (PE); and
comparing the level of the at least one biochemical obtained with the reference level of the biochemical of subjects known to not have ADHD, to determine whether the obtained level is different from the reference level.

2. The method of claim 1, wherein the spectral data is obtained by 2D COrrelated SpectroscopY (2D COSY).

3. A method for monitoring the effectiveness of treatment of a subject having attention-deficit/hyperactivity disorder (ADHD), comprising:
obtaining spectral data of the brain of a subject for at least one of the following biochemicals:
glutathione,
glutamine cysteine moiety,
glycerophosphocholine,
phosphoryh choline (PC),
L-isoleucine,
fucose α (1-6) glycans,
fucose α (1-2) glycan,
A phophoethanolamine,
glucose, and
Polyethylene (PE); and
comparing the level of the at least one biochemical obtained with the reference level of the biochemical of subjects known to not have ADHD, to determine whether the obtained level is different from the reference level.

4. The method of claim 3, wherein the spectral data is obtained by 2D COrrelated SpectroscopY (2D COSY).

5. A system for enabling detection of a subject to have attention-deficit/hyperactivity disorder (ADHD), comprising:
A spectroscopy device for obtaining spectral data of the brain of a subject for at least one of the following biochemicals:
glutathione,
glutamine cysteine moiety,
glycerophosphocholine,
phosphoryh choline (PC),
L-isoleucine,
fucose α (1-6) glycans,
fucose α (1-2) glycan,
A phophoethanolamine,
glucose, and
Polyethylene (PE); and
a processor for comparing the level of the at least one biochemical obtained with the reference level of the biochemical of subjects known to not have ADHD, to determine whether the obtained level is different from the reference level.

6. The system of claim 5, wherein the spectral data is obtained by 2D COrrelated SpectroscopY (2D COSY).

7. A system for monitoring the effectiveness of treatment of a subject having attention-deficit/hyperactivity disorder (ADHD), comprising:
obtaining spectral data of the brain of a subject for at least one of the following biochemicals:
glutathione,
glutamine cysteine moiety,
glycerophosphocholine,
phosphoryh choline (PC),
L-isoleucine,
fucose α (1-6) glycans,
fucose α (1-2) glycan,
A phophoethanolamine,
glucose, and
Polyethylene (PE); and
a processor for comparing the level of the at least one biochemical obtained with the reference level of the biochemical of subjects known to not have ADHD, to determine whether the obtained level is different from the reference level.

8. The system of claim 7, wherein the spectral data is obtained by 2D COrrelated SpectroscopY (2D COSY)
